(19) United States
(12) Patent Application Publication
(10) Pub. No.: US 2016/0158240 A1
(43) Pub. Date: Jun. 9, 2016

(54) Compositions Containing Defined Caffeine and Theobromine Levels with Enhanced Cognitive Properties

(71) Applicant: The Hershey Company, Hershey, PA (US)

(72) Inventors: Stephen CROZIER, Hummelstown, PA (US); Malathi NAIR, Hershey, PA (US); Heather M. ARENTZ, Hummelstown, PA (US)

(21) Appl. No.: 14/958,634

(22) Filed: Dec. 3, 2015

Related U.S. Application Data
(60) Provisional application No. 62/088,367, filed on Dec. 5, 2014.

(51) Int. Cl. A61K 31/522; A23G 1/32; A23G 1/56; A61K 31/352; A61K 36/185

(52) U.S. Cl. CPC A61K 31/522; A61K 31/352; A61K 36/185; A23G 1/56; A23G 1/32; A23V 2002/00

(57) ABSTRACT

The invention includes compositions and methods for improving cognitive function or reducing the anxiety effects of caffeine cravings. Caffeine and theobromine are naturally occurring and are both present in cocoa-derived food products or ingredients. The main substances used in the methods and food products of the invention comprise compounds known to influence the brain—caffeine and theobromine. When present in defined levels per serving, the caffeine and theobromine containing compositions of the invention result in surprising characteristics that can reduce the reliance on caffeine for habitual users, improve blood flow in the brain, and improve cognitive function.

### REFERENCE TO RELATED APPLICATIONS

This application claims priority benefit of U.S. provisional application 62/088,367, filed Dec. 5, 2014, and the entire contents of this prior application are incorporated herein by reference.

### FIELD OF THE INVENTION AND INTRODUCTION

The invention relates to functional beverages and food products containing specific levels of or relative concentrations of caffeine and theobromine, or defined amounts of caffeine and theobromine per dose or serving. Caffeine has been used and reported to increase cognitive performance, such as memory and learning performance. Caffeine has also been reported as improving alertness, mood, and concentration and focus. However, the long term use of caffeine usually results in caffeine cravings, often associated with feelings of anxiety and a decline in mood and alertness. The examples and descriptions here show that, surprisingly, a combination of caffeine and theobromine delivered in place of just caffeine or coffee can reduce the occurrence of caffeine cravings and alter blood flow to regions of the brain associated with caffeine's effects. Thus, a food or beverage prepared with a defined amount or specific relative concentrations of caffeine and theobromine and according to the invention can advantageously provide the positive benefits associated with caffeine while avoiding the negative side effects of caffeine craving.

### RELEVANCE OF THE INVENTION AND DESCRIPTION OF RELATED ART

An increasing number of published reports show the cognitive benefits of caffeine. In addition, reports discuss the use of theobromine and more generally methylxanthene compounds from cocoa as beneficially effecting cognition (H. Smit, Psychopharm., 176:412-419 (2004); R. Franco, Nutrients, 5:4159-4173 (2003)).

Caffeine and theobromine have similar structures but they have differential affinity for adenosine receptor subtypes (R. Smit, Handbook Experimental Pharmacology, Springer, vol. 200 pp. 201-234, 2011). Adenosine receptor antagonism may underlie the physiological effects of these compounds and differential affinity provides a mechanism whereby theobromine could work in parallel or in opposition to caffeine. Previously, it was not known whether adequate or specific quantities or concentrations of theobromine could either compete with caffeine for adenosine receptor subtypes or could preferentially bind adenosine receptor subtypes in the presence of caffeine, especially under conditions where antagonism by caffeine results in feelings of anxiety. As shown in the examples and data here, differences in adenosine receptor antagonism result in different patterns of neuronal activation or blood flow in the brain, and these differences can be visualized by functional MRI imaging.

In addition, none of the prior reports address how to reduce the known side effects of regular caffeine use. Furthermore, the prior work refers to combinations of caffeine and theobromine as being present in levels corresponding to those naturally occurring in typical cocoa products, such as dark chocolate.

### SUMMARY OF THE INVENTION

In one aspect, the invention addresses the problem of the common side effects associated with regular caffeine use. For example, caffeine can improve mental alertness and motor performance, but several published studies confirm that regular caffeine consumption is associated with increased feelings of anxiety as well as cravings. As the Figures herein show, the invention includes compositions and methods resulting in surprising changes in brain blood flow patterns and/or in cognitive tests following the consumption of beverages with various defined combinations of caffeine and theobromine. Thus, in one aspect, an objective of the invention is to provide a food or beverage containing a theobromine-containing cocoa extract specifically for the purpose of attaining the ergogenic or cognitive benefits of caffeine, but without the adverse feelings of anxiety associated with regular caffeine consumption and caffeine cravings. The beverages and food products of the invention are superior to other food or beverages, such as various concentrations of chocolate, cocoa, cocoa extracts comprising cocoa solids, and green coffee extracts, as they exhibit, for example, a measurable or detectable improvement in blood flow to specific regions of the brain. Furthermore, in some aspects, the levels of theobromine, about 200 mg to 250 mg per serving, would require a large amount of additional natural products or other components in order to reach these theobromine levels, making chocolate, for example, an impractical source. Accordingly, the beverages of the invention and the methods of using them provide both surprising and advantageous results compared to similar prior compositions.

In addition, differences in adenosine receptor antagonism result in different patterns of neuronal activation in the brain that can be visualized by functional MRI as shown in the examples and data provided here. This data also supports the surprising and advantageous results of using the compositions or beverages of the invention. Without limiting the scope of the invention to any particular hypothesis or method of action, the benefits of the compositions of the invention include the differential effects on adenosine receptor antagonism, where theobromine competes with caffeine for the adenosine receptor subtypes associated with caffeine cravings, or theobromine preferentially binds adenosine receptor subtypes associated with the calming effect of caffeine in regular users. Thus, the invention includes compositions and the use of compositions that differentially effect or influence adenosine receptor subtypes. These effects can be detected, measured, and/or visualized by functional MRI (fMRI) analyses over time.

In another aspect, the invention furthers the understanding of the brain's functional reaction to caffeine ingestion based upon previous studies on the analysis of the connectivity matrix visible from brain fMRI imaging. For example, following existing protocols, such as that shown in Hayasaka and Laurenti, Neuroimage, vol. 50:499-508 (2010), using a regression analysis on spatially normalized brain images, the defined combinations of caffeine and theobromine of the invention demonstrate differential connectivity profiles in regions of the brain associated with the negative aspects of caffeine cravings. The Figures show this. For example, FIG. 1 shows the typical reduction in blood flow/connectivity after a subject that routinely ingests caffeine is given a caffeine dose (“Caffeine”) to reduce caffeine cravings, whereas the “Placebo” subject still displays high levels of blood flow/connectivity in regions of the brain consistent with caffeine cravings. Comparing FIG. 3 to FIG. 1, the “Coffee-free plus Cocoa Extract” treated subject of FIG. 3 ingested a composition of the invention with high levels of theobromine, and the reduction in blood flow/connectivity in this subject is surprisingly improved even over the subject ingesting “Green Coffee” or any other test composition, and the improvement is in the same regions where caffeine cravings show negative blood flow/connectivity. In the brain images of these FIGS. 1 and 3, yellow/red areas are correlated to a negative change in blood flow/connectivity by analysis of fMRI over time, whereas blue/green areas represent a positive change. The placebo image of FIG. 1 and the coffee-free image of FIG. 3 are similar or comparable, whereas the caffeine image of FIG. 1 and “coffee-free plus cocoa extract” (high theobromine content) image of FIG. 3 are similar or comparable. The grey areas represent no significant change.

Thus, the use of compositions of the invention with defined combinations of caffeine and theobromine can address physiological conditions in the brain of animals, and specifically can reduce caffeine cravings, perhaps even better than caffeine alone. Accordingly, the invention provides compositions that allow the positive benefits of caffeine use without administering high levels of caffeine.

While the Examples and Figures show and discuss levels of improvement for the specific levels of caffeine to theobromine present, the levels or ratios of these compounds need not be exactly as described in the Examples. However, in the most preferred embodiments, the invention does not include compositions or the use of compositions where the ratio or level of caffeine to theobromine is the same as those present in naturally occurring cocoa samples, such as cocoa nibs or cocoa powder or other typical staple products of chocolate manufacture, or of any previously made chocolate beverages or compositions. In preferred examples, the amount of theobromine per serving is about 200 to 250 mg and the amount of caffeine is lower, but the caffeine present is not the same as the theobromine:caffeine ratio present in a sample of any naturally occurring product. The levels of theobromine and caffeine per serving in the compositions of the invention can be adjusted by either increasing the levels of one or the other compound present in a single dose. Thus naturally occurring cocoa samples can be used as a starting material where levels of caffeine, for example, are increased by adding caffeine. In one instance, a water extract of cocoa powder can be used as a starting material, and additional caffeine added to arrive at a desired level or ratio that differs from that present in natural cocoa. In other preferred embodiments of the invention, the compositions do not include any dairy products or milk products. A measured or detectable improvement in blood brain flow levels can indicate the desired or optimum level or ratio of caffeine to theobromine for a particular composition, a particular treatment protocol, or a particular clinical result. The term “improvement” and its grammatical variations are not intended to require an exact change in the cognitive ability or blood flow or connectivity results.

In more general aspects, the invention includes a theobromine and caffeine composition suitable for oral administration. In a preferred embodiment the composition contains (in a single serving) 250 mg of theobromine in the form of a cocoa extract and 23 mg of caffeine, the caffeine at least partially derived from the same cocoa extract. Either or both of added theobromine or added caffeine, not necessarily from any particular source, can be used to arrive at a defined level of caffeine and theobromine for a particular use. The serving is preferably a liquid or suspension, but could also be in a solid form. The level of caffeine per serving is below that present in coffee and caffeinated energy beverages, such as below 95 mg per serving. Any additives or other ingredients used in the compositions of the invention do not change the available levels of caffeine and theobromine present in a single serving. Changes in the amounts of theobromine and caffeine present per serving can be adjusted based upon the results of the functional MRI or brain blood flow data for particular treatments. Thus, in subjects with a regular caffeine or coffee consumption habit, the improvements as shown in FIG. 3 and discussed above indicate surprising results that can ameliorate or reduce caffeine craving side effects and at the same time reduce the amount of caffeine ingested.

In another general aspect, the invention includes treatment or administration methods or protocols to reduce the amount of caffeine ingested, or reduce the caffeine craving side effects or feelings of anxiety. These methods encompass the administration of the theobromine and caffeine compositions noted above at one or more times per day. In this and other related aspects of the invention, the patient population or intended subjects for administration of the compositions of the invention have a regular caffeine intake of about or more than 400 mg daily, or more than 500 mg daily, or between 200 to 500 mg of caffeine daily, or between about 400 to 500 mg of caffeine daily. Similarly, the invention includes ingestible products for use in a treatment for reducing the anxiety side effects of coffee craving, where the product contains in a single dose approximately 23 mg of caffeine and 250 mg of theobromine. The product can, in other embodiments, contain no other active therapeutic or nutritional supplements, no other theobromine-containing source, or substantially no other active or nutritional components that would alter or effect the anxiety-reducing, mood enhancing or other cognitive or brain blood flow benefits of a subject using the product. Alternatively, the product can contain additional therapeutic or nutritional components, such as those listed throughout this disclosure or available to one of skill in the art. The ingestible product can use theobromine in a form that was derived solely from cocoa sources, such as a cocoa bean extract. Various types of cocoa beans can be used, including raw beans, processed beans, or fermented beans, or a combination of these. These ingestible products can be beverages, as shown in the examples, or take a solid or other form.

In another general aspect, the invention includes methods of detecting an improvement in blood flow to regions of the brain associated with anxiety or caffeine cravings encompassing administering a theobromine and caffeine composition of the invention and measuring one or more of the functional MRI, blood flow analyses, or other tests mentioned or referred to in the Examples, such as cognitive tests. Similarly, the invention includes methods of treating anxiety and caffeine craving by administering a theobromine and caffeine composition of the invention. In a preferred example, the measurement of improvement is made within about two hours of administration. In addition, similar methods can be used to improve cognitive function or alertness in a subject by administering at least one daily serving of a theobromine and caffeine composition of the invention.

Throughout this disclosure, applicants may refer to texts, journal articles, patent documents, published references, web pages, and other sources of information. One skilled in the art can use the entire contents of any of the cited sources of information to make and use aspects of this invention. In particular, the references Monahan et al. (2011) J. Appl. Physiol. 111:1568-1574, and Simpson et al. (2013) Statistics Surveys 7:1-36, are incorporated herein by reference. Each and every cited source of information is specifically incorporated herein by reference in its entirety. Portions of these sources may be included in this document as allowed or required. However, the meaning of any term or phrase specifically defined or explained in this disclosure shall not be modified by the content of any of the sources. The description and examples herein are merely exemplary of the scope of this invention and content of this disclosure and do not limit the scope of the invention. In fact, one skilled in the art can devise and construct numerous modifications to the examples listed below without departing from the scope of this invention.

### BRIEF DESCRIPTION OF THE DRAWINGS

The following figures are examples of the scope and content of the invention and are not meant to limit the claims to any particular aspect or embodiment of the invention. The patent or application contains at least one drawing executed in color. Copies of this patent or patent application publication with color drawings will be provided by the Office upon request and payment of the necessary fee.

FIG. 1 depicts exemplary connectivity and brain blood flow analyses using fMRI as described in previous reports. Subjects with a caffeine habit are administered a placebo or caffeine as labeled. The colors further to the yellow/red end shown in the scale at bottom indicate a negative connectivity result, whereas colors on the blue/green end of the color scale indicate a positive result. Grey areas show no significant change.

FIG. 2 depicts an exemplary close-up image of the functional brain network analyses, or connectivity analysis, showing the imaged connections in the fine structure of the brain region as described in the Examples.

FIG. 3 depicts exemplary comparative connectivity analyses using fMRI showing brain blood flow differences for subjects with a caffeine habit treated with various samples as labeled and described.

FIGS. 4 and 5 are Tables showing the cognitive effects and mood responses of the treatments, as described and indicated in the examples herein.

FIGS. 6 to 8 are a graphically representation of the statistical data from the examples described here, which show the self-reported anxiety level comparison, the changes in testing accuracy comparison, and the changes in error omissions comparison. FIG. 6 depicts the mean change from baseline scores in self-reported anxiety levels with the caffeine alone composition compared to the cocoa+caffeine (Caffeinated Cocoa) composition. The FIG. 6 results show the statistically significant condition change over time. At the 93-119 mins post treatment time period, a large standardized difference of 0.84 between the two treatments exists, demonstrating that the cocoa+caffeine treatment attenuates the anxiety provoking effects on cognitive testing after caffeine alone. FIG. 7 depicts the statistical summary of the accuracy testing and FIG. 8 the statistical summary of the omission errors testing. There is a large standardized difference of 0.94 for the accuracy tests, and a moderate standardized difference of 0.50 fpor the omission errors tests at the final time point of 93-119 mins post treatment. These results follow and support the effects shown in FIG. 6 and show the benefits of the cocoa+caffeine composition over caffeine alone in improving the Bakan test results in both increased accuracy and reduced errors of omission.

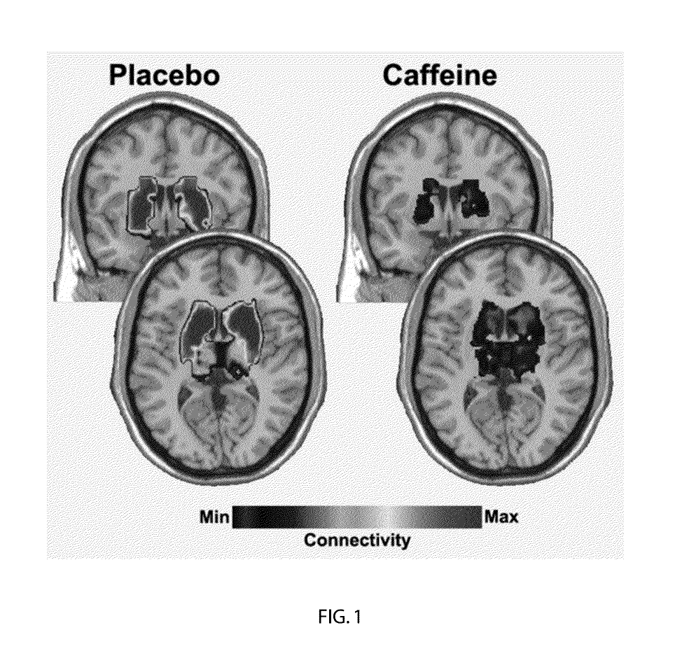

### DETAILED DESCRIPTION OF EXEMPLARY EMBODIMENTS

The invention encompasses compositions used in and methods of improving cognitive function in a subject as measured by improved alertness, brain network connectivity analysis, or other improvement in function, behavior, or mood. In a preferred aspect, the invention comprises a food product having in a single dose containing approximately 250 mg of theobromine and approximately 23 mg of caffeine. The levels of theobromine and caffeine can be designed to be different from any levels shown to be present in nature, including different from any cocoa or cocoa bean-derived staple product used in chocolate or food manufacturing. Examples of the variation in levels of caffeine and theobromine specifically include the use of cocoa extracts designed to retain high levels of theobromine, where additional caffeine is added. In these examples, the levels of caffeine and theobromine can be different from the 23 mg and 250 mg noted above. In preferred embodiments where the levels differ from 23 mg and 250 mg, one or both of the caffeine or theobromine levels can be changed by 3% or 5% or 7% or 10% or 15% or 20% or 25%, either higher or lower for each compound. Specific additional examples include 20 mg of caffeine and 220 mg of theobromine and 22 mg of caffeine and 250 mg of theobromine. Generally, compositions with about or greater than 200 mg of theobromine per serving are desirable. Thus, a variety of compositions containing defined levels of caffeine and theobromine per serving or dose are contemplated by this invention, as well as the use of these various compositions to improve cognitive function, reduce the anxiety levels of caffeine cravings, and improve mood and performance. The testing described below and referred to in the studies and the references noted can be used to optimize a particularly desired result for the use of a composition of the invention.

Previous research on caffeine has shown that approximately 17 participants are sufficient to identify the cognitive effects. In addition, a study using 6 participants demonstrated a brain blood flow difference of 5.03 ml/100 gm tissue/min between caffeine and theobromine.

In an example where four test compositions, in this case beverages, are used to demonstrate the improved characteristics of the theobromine and caffeine compositions of the invention, the beverages are: chocolate flavored beverage (placebo); chocolate flavored with green coffee; cocoa and chocolate in combination plus cocoa extract (with relative high theobromine content); cocoa and chocolate in combination plus cocoa extract (with relative low theobromine content). Each of the beverages contains FDA approved ingredients and the drinks are produced and bottled following FDA approved procedures. Not all drinks contain all ingredients from the exemplary list of ingredients that follows, but the participants consume each of the ingredients over the course of the study. None of the drinks contains more than 100% of the daily value for B vitamins. None of the drinks contains more than 95 mg of caffeine (the equivalent of an 8 oz cup of coffee).

Exemplary Ingredients: Water; Lowfat Milk; Sugar; Cocoa; Chocolate; Maltodextrin; 2% or less of: Theobromine from a cocoa extract; High Fructose Corn Syrup; Glycerin; Green Coffee Extract; Cocoa Butter; Corn Syrup; Cocoa Processed with Alkali; Natural and Artificial Flavor; Cellulose Gel; Milk Fat; Cellulose Gum; Soy Lecithin; Caramel Color; Lactose (Milk); Carrageenan; Vitamin B3 (Niacinamide); PGPR, Emulsifier; Potassium Sorbate (Preservative); Vitamin 6 Pyridoxine (Pyridoxine Hydrochloride); Xanthan Gum; Salt; Mono- and Diglycerides; Polysorbate 60; Vitamin B12 (Cyanocobalamin); Red 40 Color; Theobromine; Caffeine. Exemplary nutritional additives include: vitamin B complex, folic acid, niacin, niacinamide, pantothenic acid, pyridoxine hydrochloride, vitamin B2, folate, biotin, vitamin C, vitamin D, vitamin D3, vitamin E, vitamin K, cyanocobalamin, inositol, thiamine, thiamine mononitrate, calcium pantothenate, mixed tocophyerols, d-alpha tocopheryl acetate, magnesium, calcium, calcium carbonate, calcium chelate, calcium di-phosphate, calcium phosphate, iron, magnesium carbonate, magnesium citrate, magnesium oxide, magnesium phosphate, manganese chelate, manganese sulfate, potassium, potassium chelate, potassium chloride, sodium, zinc, vanadyl sulphate, chromium, chromium chloride, chromium picolinate, or chromium polynicotinate, 5-HTP, arginine, beta alanine, carnitine fumarate, citrulline malate, glutamine peptide, glycine, I-alanine, I-arginine, I-arginine hydrochloride, I-histidine, I-methionine, I-lysine HCL, L-phenylalanine, leucine ethyl ester, I-glutamine, I-isoleucine, I-theanine, I-tyrosine, phenylalanine, taurine, tri-methyl glycine, tryptophan, tyrosine, I-carnitine, I-carnosine, glutamine alpha ketoglutarate, and alpha-L-polylactate.

Furthermore, other ingredients commonly used or previously used in beverages, especially coffee, tea, or cocoa beverages, can also be added or used in compositions of the invention.

Measurement or detection of changes in the brain can be done using functional MRI (fMRI) and functional brain network analyses as known in the art. The following is an exemplary study using known techniques based upon fMRI scanning of human brains.

These examples represent an intervention study (number of subjects=24) with a randomized controlled, double-blind, placebo-controlled crossover design. The study is designed to ensure completion of all possibilities in the order of delivering the test samples, i.e. each subject is administered each of the possible test beverages in different order. An approximately equal number of men and women should be included and ethnic groups and minorities should also be included. For initial baseline testing, subjects undergo 16 hours of caffeine and chocolate withdrawal for a baseline testing and then participate in four separate brain scanning visits over 5-10 weeks. Study participants are ideally healthy adults 18-55 who consume moderate (200-500 mg) amounts of caffeine daily. Participants must withhold all caffeine and chocolate from the diet for 16 hours prior to each study visit. As determined from a medical screening session, any subjects with active neurological dysfunction (such as a major Axis I psychopathology, Alzheimer's disease, Parkinson's disease, prior history of stroke, epilepsy, or serious CNS trauma, ADHD, migraines, hypertension, diabetes, peripheral vascular disease, or taking vasoactive medications, such as antihypertensive medications) are excluded from the study. Individuals with any of the following conditions are excluded as well: color blindness; pregnant women; those diagnosed with depression; and individuals who are or potentially may be cognitively or psychologically impaired. Participants are sent home with a three-day caffeine consumption diary to be completed to ensure moderate caffeine use (200-500 mg/day). There are 4!=24 possible variations in the ordering of drinks. Participants are randomly assigned (without replacement) to one of the 24 drink orders. If a participant does not complete the entire study, a new participant will be enrolled and assigned that same randomization. Randomization order will be maintained in the study notebook. Baseline visits are conducted after the participant has withdrawn from caffeine for at least 16 hours so that baseline cognitive testing will be in the same caffeine withdrawn state as the MRI first scan visit and cognitive tests. The tests will be the same throughout and will be administered 1 hour post administration and again 3.5 hours post administration. Eligible participants will be scheduled for four MRI scanning visits (MR1, MR2, MR3, and MR4) to collect MRI scans and cognitive and physical measurements. Prior to each MR test session, participants will be required to abstain from all chocolate- and caffeine-containing foods or drinks for 16 hours. The session will begin by obtaining a set of vital signs followed completion of 2 mood surveys (POMS and the physical symptom survey) then the consumption of a study beverage.

Participants will consume 3 ounces of the beverage (one serving) within 10 minutes. MR scanning will begin at 1 hour post-beverage. Test sessions will be composed of a 30-minute MRI scan followed by cognitive and mood testing that will take about 30 minutes. Cognitive and mood testing will be repeated 3.5 hours post-beverage. Each testing day will be separated by approximately 10-30 days. All testing will be performed at approximately (within 2 hours) the same time on each test day. The first MR visit will be scheduled within 30 days of a baseline visit and continue with four study MRI visits.

Vital signs (blood pressure, pulse, respiratory rate) will be taken before as well as at 1 and 3 hours after consuming the test beverage on each testing day. A physical symptom survey will be administered immediately after the collection of vital signs. This survey has previously been used to assess caffeine withdrawal symptoms (Addicott and Laurenti, Psychopharmacology vol 207(3):423-431 (2009)).

The following brain imaging data can also be collected: Anatomic imaging—a structural brain image can be important for data analyses and for data interpretation; Cerebral perfusion—a non-invasive measure of whole brain blood flow; Resting fMRI—fMRI scans will be used to evaluate whole-brain network connectivity and brain network analyses to show how brain regions work together as an integrated system.

Cognitive tests to be performed on the subjects can include those know in the art, including: the n-back test is a measure of working memory; the Hopkins Verbal Learning Test (HVLT) assesses episodic memory, with versions 1-6 differing in only in the words used for testing; Reaction time task to measure sensory-motor transformations and speed of response; the Eriksen flanker task evaluates executive function and spatial attention; the Stroop Task measures response inhibition, conflict resolution, and executive function; and the Profile of Mood States (POMS) is a short, multiple choice test to self-report a measure of mood states.

Measurements of Results—fMRI

MRI Brain Scanning.

Images are collected with a 3T Siemens scanner using a 32-channel head coil. High-resolution T1-weighted structural scans are obtained using an inversion recovery 3D spoiled gradient echo sequence (matrix size=256×256; field of view=24 cm; 1.5 mm sections, no gap; 128 slices; in-plane resolution=0.94 mm). Whole-brain network connectivity is assessed using blood oxygenation level dependent (BOLD) imaging (Laurenti et al, Neurolmage vol 17:751-757 (2002)). The images are collected in parallel to the anterior commissure-posterior commissure (AC-PC) line in the brain. Images are also collected using multi-slice gradient-echo planar imaging (EPI) (TR=2000 ms; TE=40 ms; field of view=24 cm (frequency)×15 cm (phase); matrix size=96 x 86, 40 slices, 4 mm thickness, no skip; voxel resolution=4×4×4 mm).

Perfusion Images.

Arterial Spin Labeling (ASL) MRI to acquire whole-brain resting baseline cerebral blood flow (CBF) can be performed with a pseudo-continuous ASL (PCASL or pCASL) sequence (XU, et al. NMR Biomed. Vol 23(3):289-293 (2010)). The PCASL sequence uses RF pulse trains and gradient fields to induce flow driven adiabatic inversion of the magnetization of arterial blood. PCASL offers higher a signal to noise ratio and a well-controlled temporal bolus, leading to robust CBF quantification compared to pulsed ASL. In addition, PCASL can be implemented without the need for a special RF system, which is often required for continuous ASL. Scan parameters can be: tagging duration=1.5 sec; TI=3 sec; TR=4 sec; repetitions=81 (40 tag/control pairs, the first image is for reference magnetization); FOV=24×24 cm; matrix size=64×64, 24 5 mm axial slices; a single shot EPI acquisition with GRAPPA factor of 2; and acquisition time=5:36 minutes.

Functional Brain Networks.

Resting BOLD fMRI images will be collected and processed using FSL unix-based software known in the art. To correct for motion, functional data sets will be realigned in using the first image as the reference. Data will then be normalized, for example to Montreal Neurological Institute (MNI) space. Global values for the whole brain, white matter, and cerebrospinal fluid will be regressed from the time series to remove spurious signals associated with physiological noise such as heart beat and respirations. The first step in performing the-network analysis is to generate a whole brain connectivity matrix, or adjacency matrix (Aij). The determination of a connection between i and j will be performed using regression analysis on spatially normalized brain images (Hayasaka and Laurenti, Neurolmage vol 50(2):499-508 (2010)). The full regression analysis will be performed for each node with all other nodes to produce a partial correlation coefficient matrix. A threshold will then be set to dichotomize the data resulting in the binary adjacency matrix (Aij). This results in a binary n×n matrix, where n=the number of voxels in the fMRI data (˜20,000). The threshold will be set based on recent work showing a universal relationship between node number and density (Newman, SIAM Review vol 45:167-256 (2003)).

Network Metrics: Once a complete adjacency matrix is generated, the characteristic network attributes can be calculated for each node or voxel in the brain. The data allow one to identify global and local changes in network topology. Whole brain network metrics will be evaluated. In addition, network metrics will be mapped back into brain space to identify spatial patterns. The primary analysis for brain networks will focus on the modular structure of the basal ganglia.

In a placebo-controlled, double-blinded, cross-over study to show the effects of two different 52 oz of ground cocoa to produce brewed cocoa compositions, a first beverage (“cocoa” containing 21 mg caffeine, 179 mg theobromine, 499 mg flavanols and natural and low calorie sweeteners) and a second beverage (“cocoa+caffeine” containing 70 mg caffeine, 179 mg theobromine, 499 mg flavanols and natural and low calorie sweeteners) can be conducted over different time periods. Tests before consumption and 21-47, 57-83 and 93-119 minutes post-consumption can be conducted. A control beverage can be used to reduce potential null findings, and a “caffeine-only” composition is used as control (52 oz of brewed cocoa water containing 66 mg caffeine, caramel coloring and natural and low calorie sweeteners) and matched to the cocoa+caffeine composition. To document whether the participants are responsive to a stimulus known to alter motivation, mood and/or cognitive performance such that the caffeine-only composition is a positive control. The fourth treatment composition can be a placebo containing neither cocoa nor caffeine and consisting of 52 oz of brewed water, caramel coloring and natural and low calorie sweeteners.

Potential participants are screened using questionnaires (medical history, diet, daily caffeine consumption, Profile of Mood State, etc.). Excluded subjects are those whose body mass index was greater than 30 or who reported: (i) an allergy to cocoa, chocolate, or caffeine, (ii) any smoking, or (iii) above average feelings of energy (scores>12) during the week prior to the screening using the vigor scale of the 30-item Profile of Mood States questionnaire (Lorr et al., 2003, Profile of Mood States, Toronto, Canada, Multi-Health Systems). Potential participants are also excluded because of over-the-counter and prescription medication use (except for contraceptives) or high consumption of flavanols during the prior month (>39 total combined servings of cocoa, caffeine, fruits or vegetables high in flavanols) using medical history and diet questionnaires described previously. A total patient of subject population of 24 is identified (18 women and 6 men). The final sample population size of 23 can be used with an age range of 18-29 years with a mean±SD age (20.25±2.23 yr), height (168.28±1.19 cm), weight (67.05±14.87 kg) and BMI (23.26±3.84 kg/m2).

The number of hours of reported sleep on a typical night during the month prior to the study was 7.4±1.1 hours. The number of hours of reported sleep the night before each of the 4 testing sessions did not differ significantly (p=0.767); for placebo (7.64±1.38 hours), for caffeine (7.92±1.38 hours), for cocoa (8.03±1.14 hours), and cocoa+caffeine (7.93±1.31 hours).

On average, participants consume 25±8 servings of foods or beverages high in flavanols during the month. Participants are also low consumers of flavanols based on the following average number of monthly servings of caffeinated drinks (0.79±2.25), cocoa (2.88±2.29), fruits (4.13±3.1) and vegetables (14.88±6.53).

Salivary Caffeine, Theobromine and Paraxanthine Levels

Saliva samples are obtained by passive drool using the SalivaBio collection system (Salimetrics, State College, Pa., USA). Samples are collected at the start of each day's testing in order to confirm compliance with the instructions to avoid cocoa and caffeine containing foods and beverages. Post-test session saliva samples are obtained to explore the association between changes in selected methylxanthines and changes in mood and cognitive performance. The saliva samples were frozen at −80° C. After all samples were collected, they were stored in dry ice for analysis. The samples were analyzed for theobromine, caffeine and paraxanthine with liquid chromatography-tandem mass spectrometry using previously described methods (Ptolemy et al., 2010, J Chromatogr B Analyt Technol Biomed Life Sci 878: 409-16).

Mental Energy Test Battery

The mental energy test battery is comprised of self-reported motivation and mood measures and computerized cognitive tasks of sustained attention. These measures are chosen because they are consistent with a model of mental energy developed for nutrition researchers (O'Connor, 2006, Nutr Rev 64: S2-6). In addition, two serial subtraction tasks (Serial Threes and Serial Sevens) are included to facilitate comparisons with prior research on cocoa that used these tasks (Scholey et al., 2010, J Psychopharmacol 24: 1505-1514).

All cognitive testing is performed in a seated position in a thermoneutral (23±1° C.), sound-attenuated (˜60 dB(A) below ambient), chamber with lighting at ˜80 lux. Visual stimuli are presented that required a finger response. Participants used either the keyboard (questionnaires and subtraction tasks) or a key pad (RB-530 key pad, Cedrus, San Pedro, Calif., USA) to respond to information presented on a 20″ computer monitor. Prior to each cognitive task the participants are given on-screen instructions and asked either to press the enter key if they understood the directions or to get help from the researcher if they were uncertain. The Continuous Performance Task and the Bakan test were scored using Cedrus Data Viewer.

1) Serial Three and Serial Seven subtraction tasks. Participants are required to count backwards in threes or sevens from a random starting number between 800 and 999 that is presented on the computer screen. Participants typed a response as quickly and as accurately as possible. The number is cleared after the entry of the first response or after three seconds. The task is scored for the number of correct and incorrect responses and the total time to complete the test. In the case of incorrect responses, subsequent responses are scored as correct if they are correct in relation to the new number. Participants are given an opportunity to complete 45 responses and are given 3 seconds for each keystroke response before the answer is marked incorrect. The answers that are marked incorrect due to no response are classified as omission errors.

2) Continuous Performance Task. Participants monitor a continuous series of letters presented on the screen for 1000 ms. The aim is to respond to the detection of the letter “X” only when it was preceded by the letter “A” by striking the left key on the key pad. The task is scored for percentage of target strings correctly detected, errors of omission (missed targets), average reaction time for correct detections, and the number of false alarms. The task lasts for 2 minutes and 48 targets are presented.

3) Bakan task. Participants are required to monitor a continuous series of digits (1-9; Tahoma Regular font, size 20). Each individual digit is presented for 1000 ms and the participant is given a primary and secondary task. The participant's primary task is to detect the presentation of three successive odd digits that are all different (e.g., 9-3-7), and the secondary task involved the identification of a specific number (i.e., 6). The participants press the right key for primary responses and the left key for secondary responses. The task is scored for the number of primary and secondary targets correctly detected, the average reaction time for correct detection of each target, the number of false alarms for each task, and errors of omission (missed targets). The task lasts 16 minutes and a total of 960 stimuli are presented during that time. There are 8 primary targets and 96 secondary targets.

4) Motivation to perform cognitive tasks. Participants rate the intensity of their current motivation to perform the cognitive tasks using a scale supported by validity evidence (Maridakis et al., 2009, Int J Neurosci 119: 975-94). The scale is scored from 0-10 with the end points labeled “No motivation” (left end, scored as 0) and “Highest motivation imaginable” (right end, scored as a 10).

5) The Profile of Mood States (POMS). The 30-item brief POMS is used to measure the mood states of tension, depression, anger, vigor, fatigue and confusion (Lorr et al., 2003, Profile of mood states (poms), Toronto, Canada: Multi-Health Systems). Participants indicate the intensity of their current feelings on the five-point scale that ranged from “Not at all” (scored as 0) to “Extremely” (scored as 4).

6) Mental and physical energy and fatigue state scales. Participants rate their current feelings of mental and physical energy and fatigue using a scale supported by validity evidence (Maridakis et al., 2009, Int J Neurosci 119: 1239-58; Moore et al., 2012, J Sports Sci 30: 841-850). Energy scale items are feelings of energy, vigor, and pep and fatigue items are feelings of fatigue, exhaustion, and being worn out. Ratings are scored on a 0-10 Likert scale and anchors indicated the absence of the feelings (left end, scored as 0) and the strongest intensity of the feelings (right end, scored as 10).

Test Beverages

The participants consume one of four 16-oz beverages on each testing day. The brewed beverages are made from either 1) ground cocoa, 2) caffeinated cocoa, 3) sweetened placebo, or 4) sweetened placebo with caffeine. The beverages are brewed in a coffee maker to a temperature of ˜167° F., and then allowed to cool uncovered for 7-8 minutes. Six cups of distilled water are filtered through the coffee maker with ˜52 oz of grounds (cocoa or placebo) to produce 16 oz of beverage. The beverage is brewed after the completion of questionnaires asking about sleep and the consumption of caffeine, cocoa or medications in the last 24 hours. To keep the participants blind to the conditions, dark coloring (DDW The Colour House-product 034, Lot#201205080070) is added to the beverages to provide a uniform color. Participants also wear a nose clip during beverage consumption and a lid covered the cup while the beverage is being consumed. Participants consume the beverage within 10 minutes of being served.

A chemical analysis, performed by the Hershey Company, is provided in Table 1.

TABLE 1
Chemical analysis of the test beverages.

| | Total | Theobromine | Caffeine |
|---|---|---|---|
| Beverage | Flavanols mg | mg | mg |
| Cocoa | 499 | 179 | 21 |
| Flavored placebo | 4 | 0 | 0 |
| Flavored caffeine | 4 | 0 | 66 |
| Caffeinated cocoa | 455 | 179 | 70 |

In the Table 1 above, Total Flavanols includes monomers, oligomers, and polymers and is determined using the method described by Payne M J, Hurst W J, Stuart D A, Ou B, Fan E, Ji H et al. Determination of total procyanidins in selected chocolate and confectionery products using DMAC, J AOAC Int 2010; 93(1):89-96.)

Prior to all testing days the participants are advised to abstain from chocolate/cocoa, caffeine, and alcohol consumption and the use of all medications except for oral contraceptives for a minimum of 24 hours prior to each testing day. Participants are also advised to get a typical amount of sleep.

Familiarization Days 1-2. On Day 1, a 30-45 minute practice session is conducted in which participants completed a single trial run of all the daily assessments. On Day 2, participants complete the entire 2.75 hour protocol, including consumption of a bottled, carbonated, 100 kcal beverage as the practice treatment.

Testing Days 3-6: Four different treatment orders are used to minimize potential order effects. The participants are randomly allocated to complete one of four beverage orders (coded as 1-2-3-4, 2-3-4-1, 3-4-1-2 and 4-1-2-3) in blocks of four, such that each of the four orders is completed by six participants. There is a minimum of 48 hours between testing days. Participants are tested from 8 am until 8 pm, but each participant is tested at the same time of day (±30 minutes) to minimize potential diurnal variation. Because sleep loss has substantial effects on mood and cognitive performance, participants who reported 2 hours more or less than their usual sleep duration reported during the screening are not tested that day and rescheduled as are those who report drug use or the consumption of cocoa or caffeine-containing beverages or foods within the prior 24-hours.

Participants are asked to accumulate saliva at the bottom of their mouth and use a plastic straw to collect 1 ml of saliva into a 2 ml test tube. Baseline measures of mood, motivation and sustained attention (i.e., the mental energy test battery) are obtained. After completion of the baseline measures, participants are served the hot beverage and asked to consume it within 10 minutes. Participants are given a 20-minute break but are not allowed to participate in strenuous physical or mental activity or consume additional snacks or beverages. Three additional 26-minute mental energy battery tests are completed and punctuated by 10-minute rest breaks.

TABLE 2
The timing of the events in the testing session

| | | | Time after |
|---|---|---|---|
| Time | | Test | Cocoa |
| (minutes) | Event | Phase | Consumption |
| 0-2 | 24-hour history | Baseline | NA |
| 2-10 | Saliva sample | Baseline | NA |
| | obtained | | |
| 10-36 | Mental energy | Measurement | NA |
| | test battery | Time 1 | |
| 36-46 | Beverage | Treatment | NA |
| | consumption | | |
| 47-66 | Rest break | Rest 1 | 1-20 minutes |
| 67-93 | Mental energy | Measurement | 21-47 minutes |
| | test battery | Time 2 | |
| 93-103 | Rest break | Rest 2 | 47-57 minutes |
| 103-129 | Mental energy | Measurement | 57-83 minutes |
| | test battery | Time 3 | |
| 129-139 | Rest break | Rest 3 | 83-93 minutes |
| 139-165 | Mental energy | Measurement | 93-119 minutes |
| | test battery | Time 4 | |

TABLE 3
Timing of the mental energy test battery

| Task | Approximate Times | |
|---|---|---|
| Motivation to perform cognitive tasks | 0.5 | minutes |
| Likert scales of energy and fatigue | 1 | minute |
| POMS fatigue and vigor scales | 2.5 | minutes |
| Serial subtraction of the number three | 2 | minutes |
| Serial subtraction of the number seven | 2 | minutes |
| Continuous performance task | 2 | minutes |
| Bakan task | 16 | minutes |

Data Treatment and Statistics

Preliminary analyses. Questionnaire data are downloaded into and can be summarized using Cedrus Data Viewer (Cedrus Corp, 2007). All data are exported into SPSS (Version 20) for analysis. All the statistical analyses are performed prior to breaking the blind. If an individual has cognitive task performance scores that are deemed as error-dominated outliers (>3 standard deviations from the mean, invariant responding resulting in zero correct answers on multiple days, ID 54321), the data from this individual are excluded from the primary analysis. Scatterplots and descriptive statistics are evaluated. Variables that are not normally distributed (i.e., assessed from Kolmogorov-Smirnov tests <0.05) are transformed using either a square root or log transformation prior to the primary analyses. The post-treatment minus pre-treatment changes in salivary concentrations of caffeine, theobromine and paraxanthine in the placebo, caffeine, cocoa and caffeinated cocoa conditions are examined using t-tests to examine whether the treatments influenced salivary methyxanthine concentrations in expected ways (e.g., caffeine increasing in caffeine conditions; theobromine increasing in theobromine conditions).

Participants with baseline saliva samples on 2 of 4 testing days that contained >0.5 μg/ml caffeine and paraxanthine suggesting a failure to comply with the instructions to abstain from caffeine can be excluded. When data from these participants are included, one-way ANOVAs can reveal if there is any statistically significant difference among the conditions in pre-testing salivary caffeine (p=0.50) or paraxanthine (p=0.22). If the conclusions of the investigation remain unchanged whether these participants are included or not, the data can be included in the analysis. Similarly, data showing use of drugs or prescription drugs can be analyzed to determine inclusion or exclusion.

Primary Analyses. Hypotheses are tested using a series (i.e., all outcomes variables) of two factor (2 Treatment×4 Time point) repeated measures ANCOVAs that control for the prior night's sleep time. The primary interests are the presence of statistically significant (p<0.05) interactions of time and either cocoa versus placebo, cocoa+caffeine versus cocoa, or cocoa+caffeine versus caffeine alone. Adjustments for sphericity, when needed, can be made using Huynh-Feldt epsilon. The source of significant interactions can be explored with one-way ANOVAs and t-tests with familywise error controlled using LSD post-hoc tests. Effect size is presented as η2 or Cohen's d (calculated based on the mean change over time in a treatment condition minus the mean change over the same time in the placebo condition, and this difference score is divided by the baseline pooled standard deviation).

Results. Expected changes in salivary methyxanthines can be observed. Caffeine levels increase significantly in the caffeine (mean change=5.3 μmol.L-1; t=8.676, df=44, p<0.001) and cocoa+caffeine (mean=5.0 μmol.L-1; t=9.311, df=44, p<0.001) treated patients, and caffeine levels did not differ between these two groups. Theobromine levels increase significantly in the cocoa (mean=26.2 μmol.L-1; t=11.655, df=44, p<0.001) and cocoa+caffeine (mean=28.9 μmol.L-1; t=11.232, df=44, p<0.001) treated patients and theobromine levels did not differ between these two groups. Paraxanthine levels increase significantly in the caffeine (mean=1.4 μmol.L-1; t=2.689, df=44, p=0.01) and cocoa+caffeine (mean=1.1 μmol.L-1; t=2.199, df=44, p=0.033) treated patients and paraxanthine levels did not differ between these two groups. There are insignificant changes in all of these three methylxanthines in the placebo condition.

Effects of Cocoa Versus Placebo

Compared to placebo, cocoa has significant interaction effects on both the reaction time response to the secondary targets of the Bakan test (F=2.679, df=3,129, η2=0.071, p=0.05) and the overall false alarms in the Bakan test (F=3.735, df=2.498, 107.42, η2=0.08, p=0.019). Reaction times are faster at all post-test time points after consuming cocoa compared to pre-consumption baseline (range=11-17 msec) while the comparable data after placebo are uniformly slower compared to baseline (range=4-11 msec); the post-hoc tests are not statistical significant (p>0.05). After taking cocoa, the participants averaged 1.6 fewer false alarms compared to baseline while after placebo they averaged 2.4 more false alarms compared to baseline. At post-test time 3, the size of the interaction is significant (t=2.28, df=44, p=0.05) large (d=0.76). No interactions are found for the other cognitive, mood and motivation variables.

Effects of Caffeinated Cocoa Versus Caffeine Alone

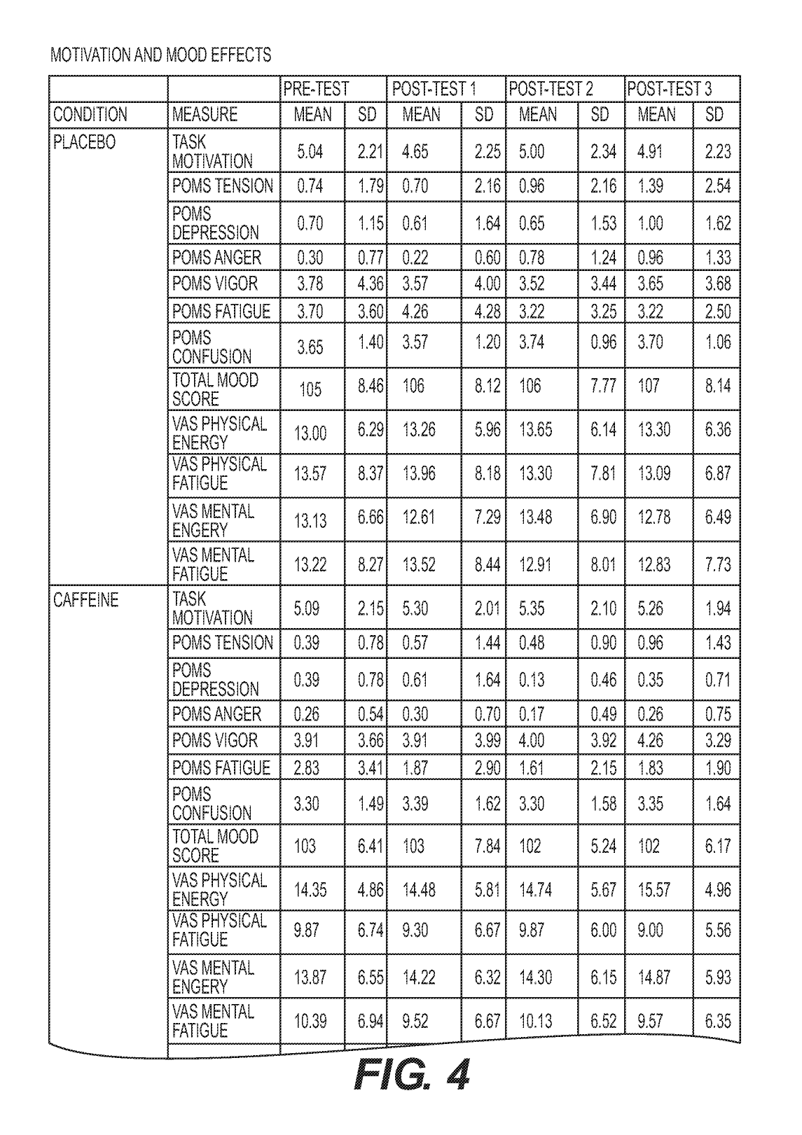

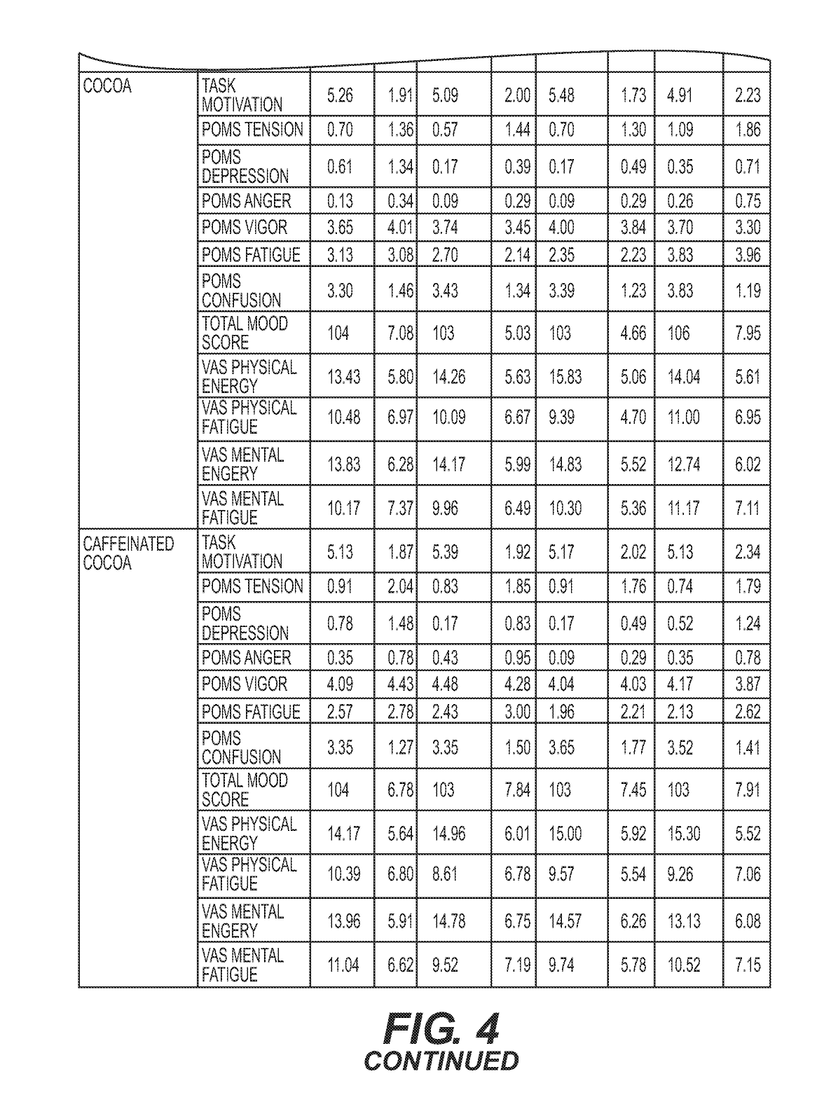

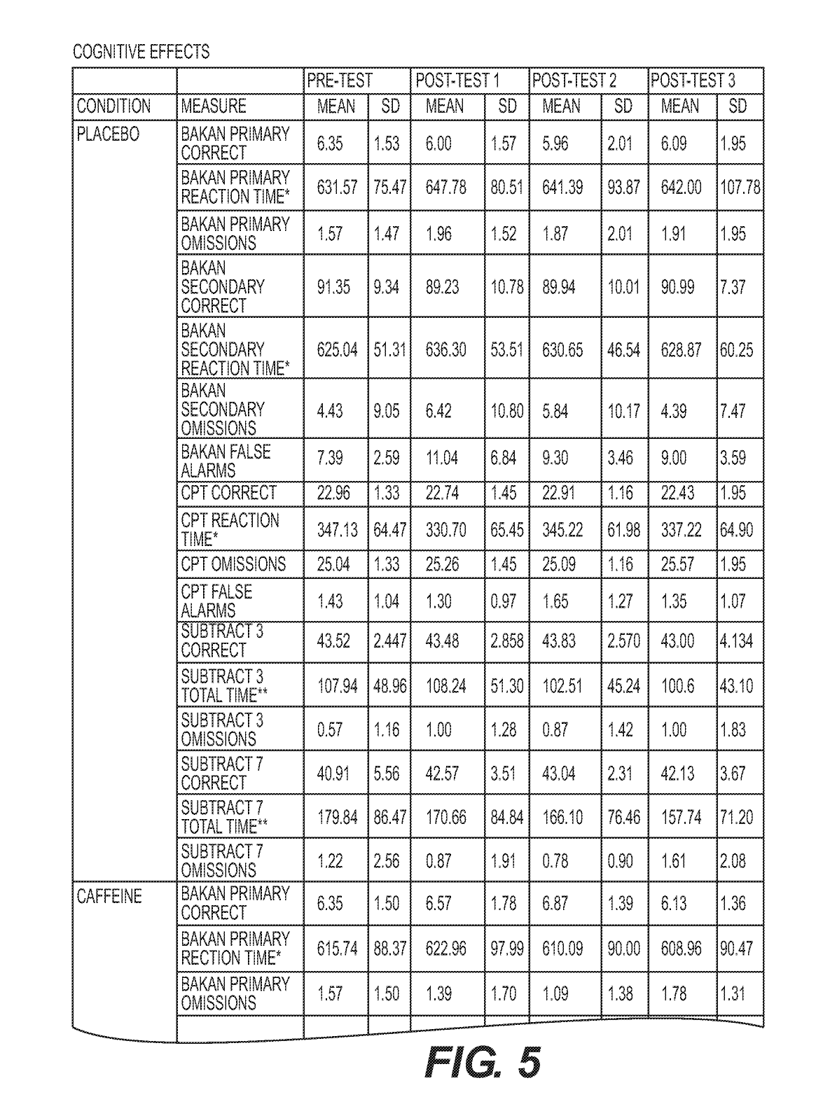

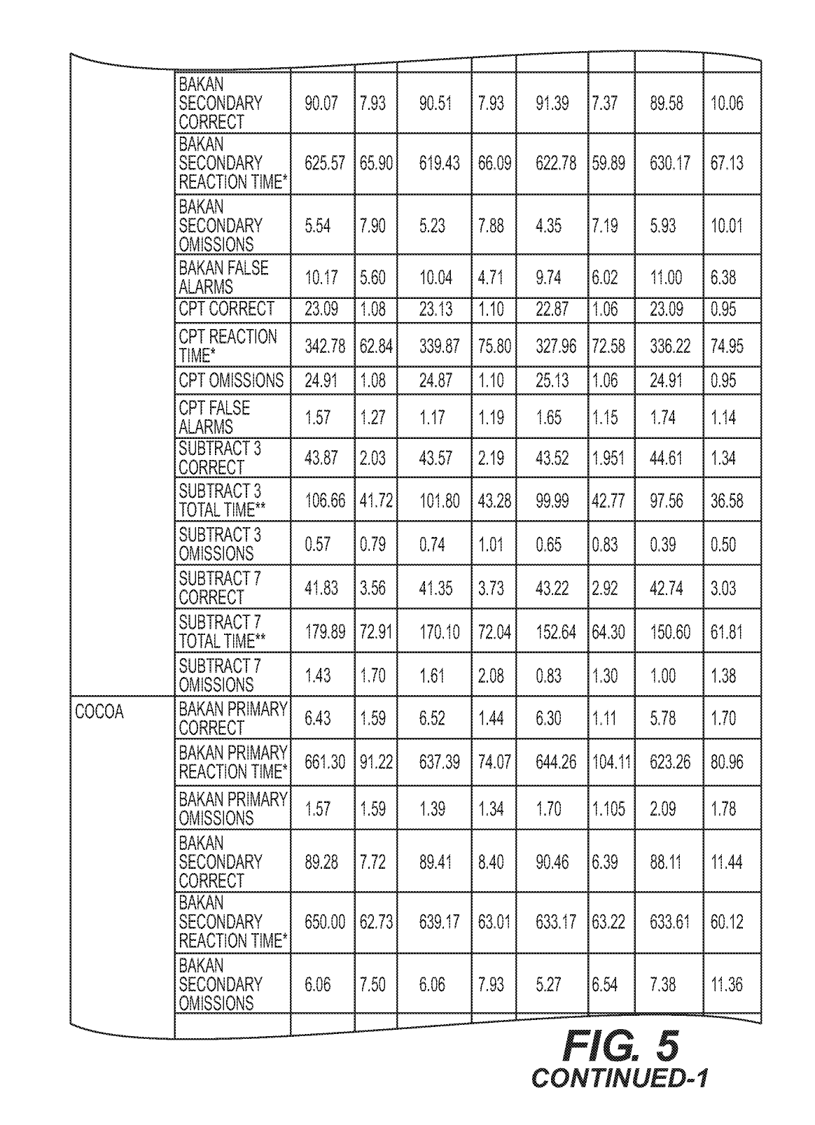

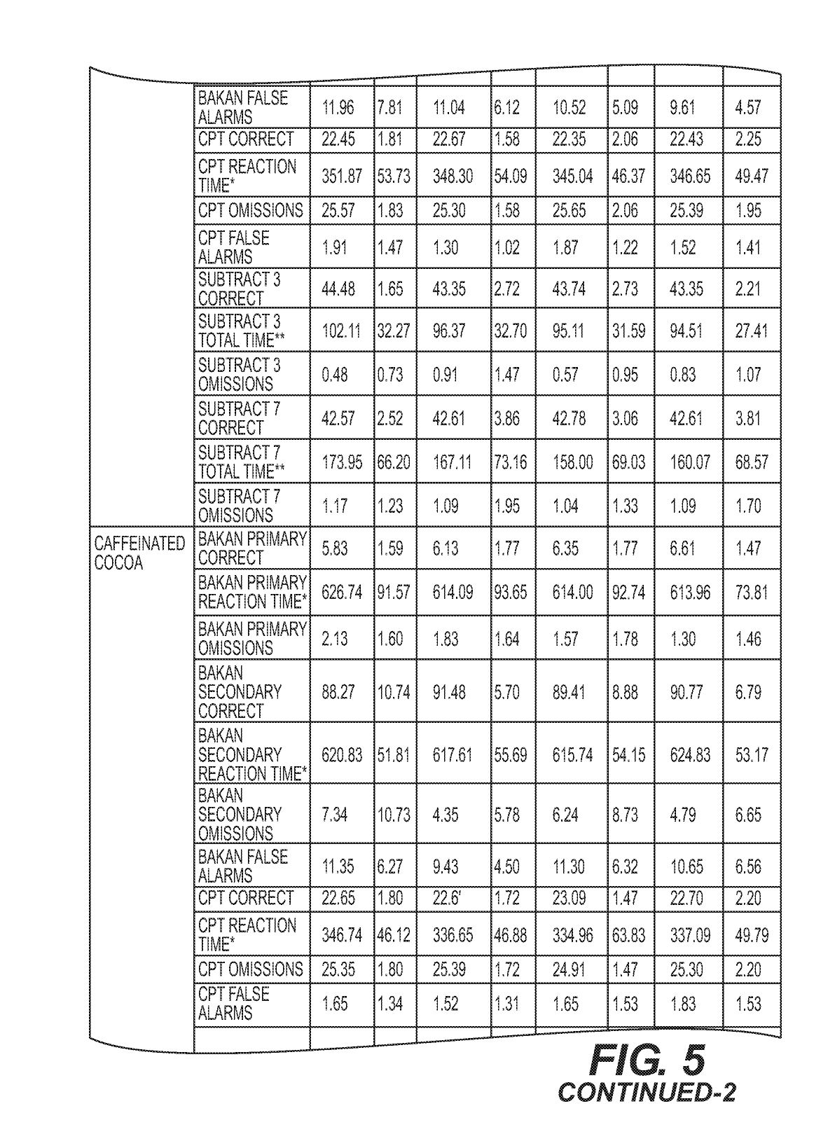

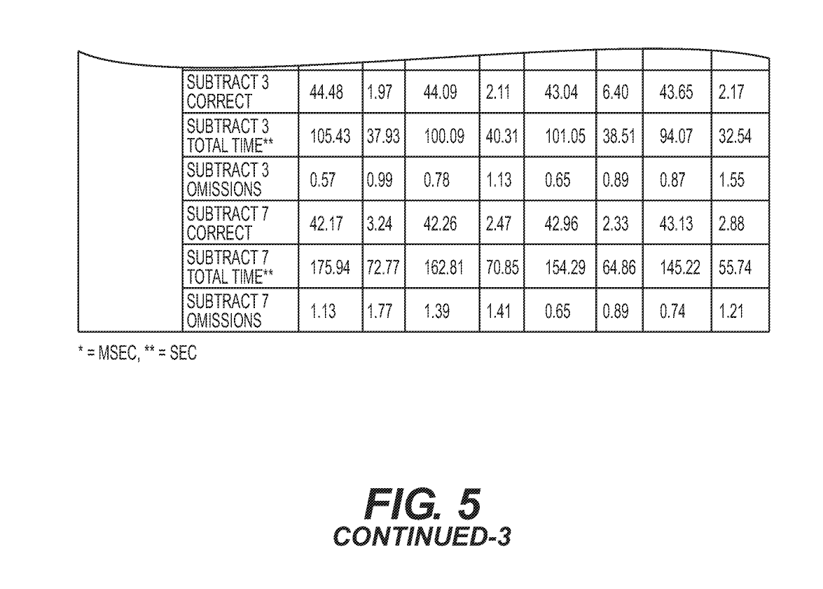

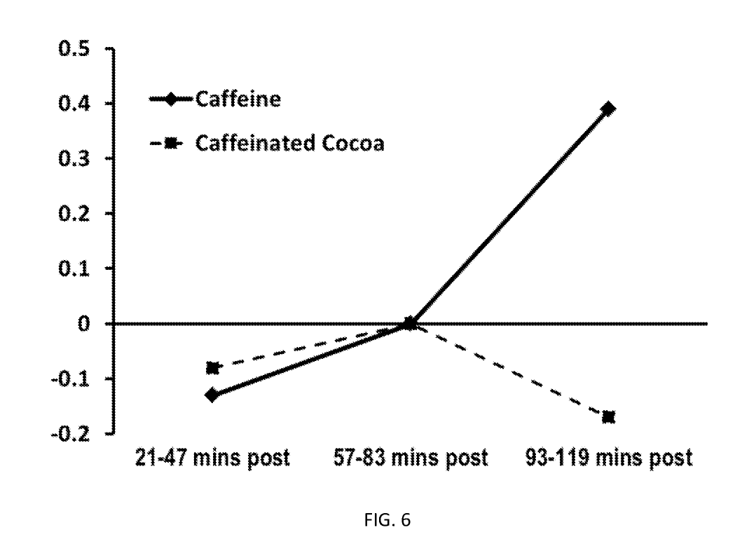

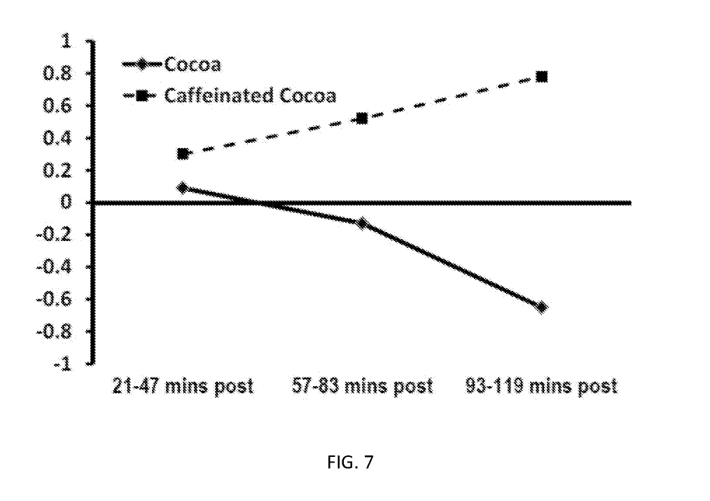

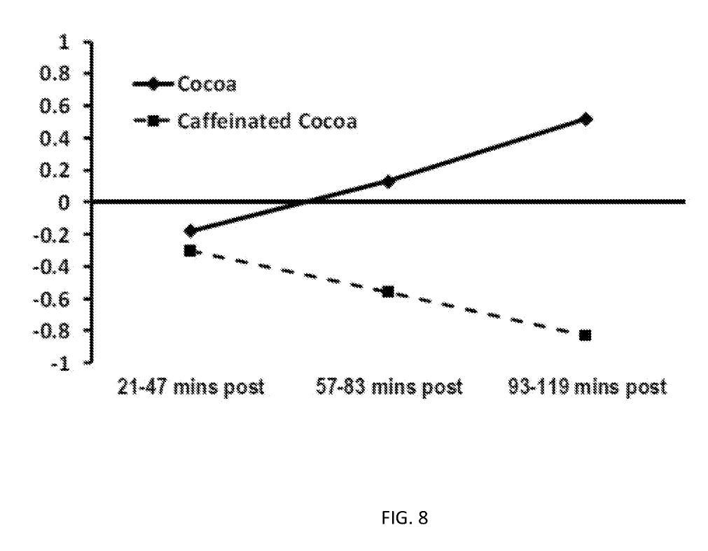

Compared to caffeine alone, caffeinated cocoa (cocoa+caffeine) has significant interaction effects on anxiety (F=2.963, df=2.8, 120.399, η2=0.064, p=0.038). These data are illustrated in FIG. 4 (Table 4). Notably, at the final testing time, anxiety levels can increase by an average of 0.57 raw score units after the caffeine alone beverage is consumed, but can decrease by 0.17 raw score units after caffeinated cocoa (cocoa+caffeine) beverage is consumed. The effect size for the difference between these two beverages/patient conditions is large (d=0.84) and statistically significant (t=2.27, df=44, p<0.05). No significant interactions are found for all other mood, motivation and cognitive variables.

Effects of Caffeinated Cocoa Versus Cocoa Alone

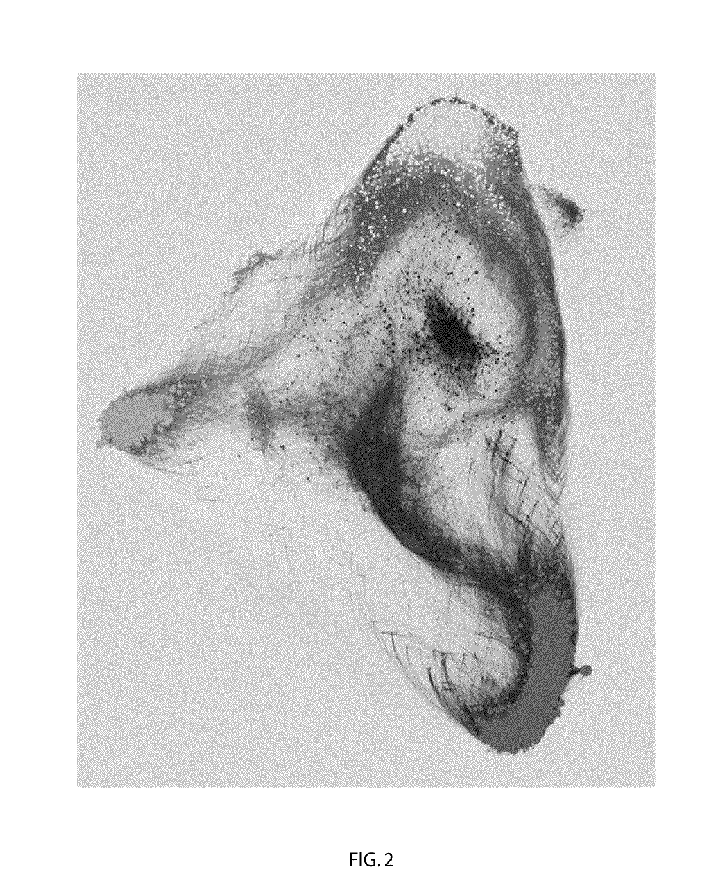

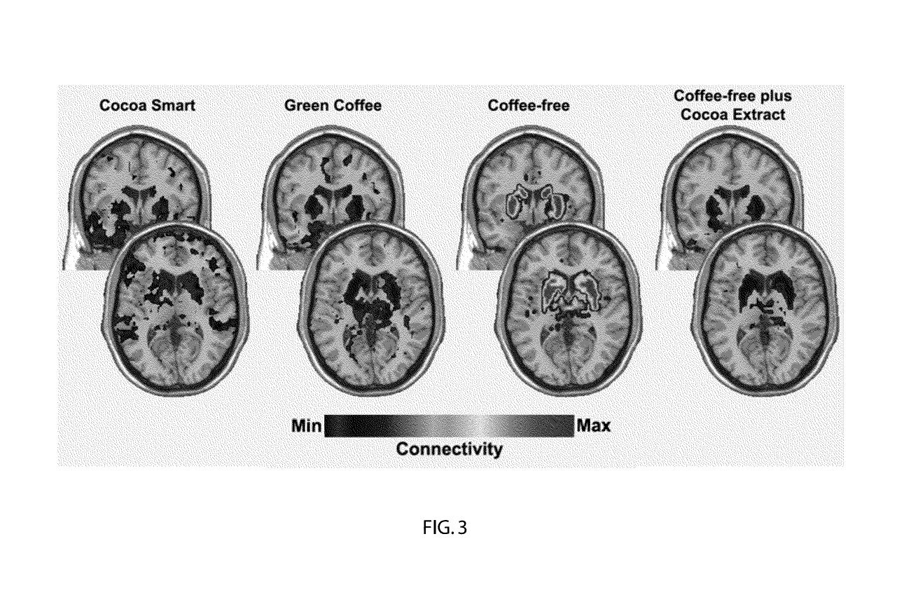

Compared to cocoa alone, caffeinated cocoa (cocoa+caffeine) has significant interaction effects on the number of correct responses (i.e., accuracy) (F=3.971, df=4.561, 1.149, η2=0.085, p=0.01) and the number of omission errors (F=3.583, df=3, 129, η2=0.077, p=0.016) for the primary Bakan task. These interactions are illustrated in FIG. 2X. The number of correct targets for the Bakan primary test steadily increase from a baseline for caffeinated cocoa, whereas with cocoa alone the number correct is below baseline at post-test times 2 and 3 after a slight increase at post-test time 1. At the final testing time the effect size for the difference between the conditions in the number of correct responses was significant (t=2.45, df=44, p<0.05) and large (d=0.94). Thus, the treatment with the caffeinated cocoa (cocoa+caffeine) also results in a steady decrease in the number of omission errors, whereas cocoa alone leads to increases. At the final testing time the size of the difference between the conditions in the number of omission errors was significant (t=2.14, df=44, p<0.05) and moderate (d=0.50). No interactions are found for all other cognitive, motivation and mood variables.

Effects of Caffeine Alone Versus Placebo

No interactions are found for all cognitive, motivation and mood variables except for anger (F=4.419, df=2.297, 98.770, η2=0.093, p=0.011). At the final testing time, anger levels increase by an average of 0.66 raw score units after placebo but are unchanged after caffeine alone beverage. At the final testing time the size of the difference between these conditions is large and significant (d=1.07; t=2.18, df=44, p<0.05).

Relationships Between Changes in Methylxanthines and Changes in Motivation, Cognition and Mood

Changes in the methylxanthines are weakly and insignificantly related to changes in motivation, mood and cognitive performance in all the treatment conditions except caffeine only. In the caffeine only condition, changes in salivary caffeine are significantly related to changes in physical fatigue (r=0.45), while changes in theobromine are positively correlated with changes in accuracy (r=0.51) and negatively correlated with changes in errors of omission (r=−0.51) in the Bakan primary task. These relationships remain significant after partialling out correlated changes in caffeine alone conditions (rpartial=0.50 and rpartial=−0.50). Changes in paraxanthine positively correlate with changes in accuracy (r=0.43) and negatively correlate with changes in errors of omission (r=−0.43) in the Bakan secondary task. These relationships strengthen after partialling out correlated changes in caffeine alone conditions (rpartial=0.58 and rpartial=−0.56).

Cocoa Versus Placebo

Cocoa enhanced two aspects of the Bakan dual task compared to placebo. Cocoa reduced overall false alarm errors progressively across time with 0.92, 1.44 and 2.35 fewer false alarms on average 31-47, 67-83 and 103-119 minutes post-consumption. Cocoa also improved processing speed during the secondary task of the Bakan dual task. The improvement in reaction time (11 msec faster) is apparent 31-47 minutes post-consumption and there is a slight additional improvement (a total of 17 msec faster) that is maintained throughout subsequent testing times. Mood states do not increase after taking cocoa alone compared to placebo, and this observation is consistent with studies that found no effect of theobromine on mood (Mitchell et al., 2011, Physiol Behav 104: 816-22) but inconsistent with prior work suggesting that higher feelings of energy can increase performance in the high-event rate component of a dual task (Matthews and Davies, 2001, Pers Individ Dif 31: 575-589).

Regression to the mean could not be ruled as an explanation for the significant effects of cocoa on the Bakan test because there are significantly (p<0.01) fewer false alarm errors (mean=4.6) and slower reaction time (mean=25 ms) at baseline in the placebo condition compared to the cocoa condition.

It is difficult to compare the Bakan secondary task results directly to other cocoa investigations because dual tasks were not used in the prior related cocoa studies (Field et al., 2011, Physiol Behav 103: 255-260; Scholey et al., 2010, J Psychopharmacol 24: 1505-1514). One prior study did not show fewer false alarms after 520- or 994-mg cocoa (Scholey et al., 2010). The failure of cocoa to significantly improve reaction time on the primary task of the Bakan test, serial three accuracy, serial seven errors, and feelings of mental fatigue were in contrast to the results of the study that is most similar in design to the present study (Scholey et al., 2010). A key difference between the present study and that study is the absence of dairy and calories in the present study compared to the dairy-based cocoa drink with ˜217 kcals used by Scholey and colleagues (2010). The Bakan test used in this study also may have different psychometric properties from the conceptually similar rapid visual information processing (RVIP) test used in the Scholey et al. (2010) study, which may have contributed to different results. For example, the reliability or the sensitivity for measuring change might differ between the Bakan and the RVIP tests because of procedural differences in the tests. The RVIP test requires participants to react to both odd and even sequences while the Bakan requires responses to odd sequences as a primary task and a single even number as a secondary task. Also, the Bakan task duration was three times longer and the stimuli in the RVIP were presented at a rate of 100 per minute while the Bakan test presented stimuli at a rate of 60 per minute. Another study using a 500-mg cocoa drink showed results that appear to be generally consistent with the present findings, but 2 of 3 testing times were confounded by the post-cocoa consumption of a lunch (Pase et al., 2013, J Psychopharmcol 27: 451-458), which reduces the ability to make meaningful comparisons to the calorie-free cocoa drink used here.

Caffeinated Cocoa (Cocoa+Caffeine) Versus Caffeine Alone

Caffeinated cocoa compared to caffeine alone allowed for an assessment of the potential role of cocoa flavanols combined with theobromine, which were both absent in the caffeine alone drink. Anxiety is the only significant interaction observed. Importantly, caffeinated cocoa shows an attenuated response to the typical increase in anxiety that occurs at the final testing time in the caffeine only condition. Elevated anxiety is a common side effect of caffeine consumption in both the low caffeine consumers, and many participants in past studies using similar protocols have anecdotally reported that repeatedly completing the sustained attention task is stressful while consuming caffeine (Maridakis et al., 2009a, Rogers, 2010). Thus, the anxiety elevation at the final testing time in the placebo condition, while not hypothesized, is not unexpected. Theobromine and flavanols, or their metabolites, could reduce or influence anxiety by binding to adenosine or benzodiazepine receptors. One study found that 500 mg cocoa acutely increased calmness; however, increased calmness did not accrue after an acute cocoa administration at the start of the investigation, but only after an acute administration was preceded by 30-days of daily cocoa supplementation (Pase et al., 2013).

Caffeinated Cocoa (Cocoa+Caffeine) Compared to Cocoa Alone

Caffeinated cocoa compared to cocoa alone allowed for a direct assessment of the impact of 49 mg of supplemental caffeine used in brewed cocoa beverages. Supplemental caffeine improves the accuracy and results in a fewer number of omission errors on the primary task of the Bakan. Improved accuracy and fewer omission errors on the primary Bakan task also occurs when the cocoa+caffeine condition is after the caffeine alone condition, but the effect was smaller. Caffeine can improve vigilance performance by improving accuracy, reducing errors and reducing reaction time (Foxe et al., 2012, Neuropharmacology 62: 2320-7; Hewlett and Smith, 2007, Hum Psychopharmacol 22: 339-50) so it is unclear why the effects of supplemental caffeine are limited to the primary task of the Bakan test. One possibility is that the participants in the present study were not especially responsive to the mood, motivation and attention enhancing influence of caffeine. Genetic factors are known to influence caffeine sensitivity and relevant genotypes, such as for adenosine A2A receptors, was not assessed in the present investigation (Rogers et al., 2010). Another possibility is that the effect of caffeine was evident only during the most challenging component of the more challenging dual task. It has been suggested that while high event tasks take more cognitive resources, low event tasks, such as the primary task of the Bakan, require greater vigilance (Parasuraman and Mouloua, 1987, Percept Psychophys 41: 17-22).

Caffeine Alone Versus Placebo

Caffeine alone resulted in small changes that were generally in the direction expected based on prior research (Einöther and Giesbrecht, 2013, Psychopharmacology 225: 251-274) but were small in magnitude and statistically non-significant. For instance, compared to pre-test, there were small, non-significant increases in motivation, feelings of energy and accuracy in the cognitive tests as well as small decreases in fatigue, errors and reaction times. Mean anger scores did not change in the caffeine condition, as is consistent with prior studies (Lieberman et al., 1987, Psychopharmacology 92: 308-312); however, a significant interaction emerged because anger increased in the placebo condition.

Possible Mechanisms

Caffeine crosses the blood-brain barrier and exerts CNS effects by antagonizing adenosine receptors. Dietary flavonoids are less well studied but experiments in rodents and pigs show that polyphenols, which flavonoids are, can traverse the blood-brain-barrier and accumulate throughout the brain (Schaffer and Halliwell, 2012, Genes Nutr 7: 99-109) and act on neural or glial cell-signaling pathways and increase cerebral blood flow (Spencer, 2010, Proc Nutr Soc 69: 244-260). One human study showed increased cerebral blood flow 2-4 hours after consuming cocoa flavanols and a subsequent study found a similar increase in elderly, except that it was delayed until 8 hours after ingestion (Sorond et al., 2008, Francis et al., 2006). Thus, while not limiting this invention to any particular mode or mechanism of action, the cognitive effects observed here can be the result of changes in brain blood flow from flavonoid intake. Adequate brain blood flow is known to be required for normal cognitive performance (Jacobson et al., 2011, Diabetologia 54: 245-255). In addition, methylxanthine treatments may have stimulated the release of neurotransmitters or neuromodulators. Increased dopamine release in the frontal, prefrontal and medial corticies is hypothesized to be part of the default mode network and known to play a role in attentional processing (Park et al., 2014). It is thought that caffeine antagonizes adenosine receptors in the basal ganglia, which is known to contribute to the modulation of the default mode network (Kaasinen et al., 2004, Tomasi et al., 2009). Increased dopamine in the nucleus accumbens also plays a role motivation and feelings of energy (Salamone et al., 2007). One study directly comparing the mood and cognitive effects of theobromine and caffeine concluded that theobromine may exert anti-anxiety effects by lowering blood pressure rather than any direct effect on the central nervous system. In short, the methylxanthines studied here potentially work via multiple, complex, interacting central and peripheral mechanisms.

In the caffeine only condition, changes in theobromine and paraxanthine were positively related to changes in accuracy and negatively related to changes in omission errors, but only in the more difficult Bakan dual task. These associations were attenuated when caffeine was combined with cocoa or when cocoa was consumed alone. The overall pattern of results suggests changes in cognitive performance and changes in salivary methylxanthine metabolites measured 2-hours after 66-mg caffeine consumption are only modestly related, task dependent and attenuated by the co-consumption of cocoa.

The correlational finding related to mood suggests that those with higher levels of caffeine 2-hours post-consumption, and hence have a slower metabolism of caffeine, also showed a greater increase in feelings of physical fatigue two hours after caffeine had been consumed. It is uncertain why a correlation of a similar magnitude did not emerge for mental fatigue also measured with a visual analog scale (r=0.12) or fatigue measured with the Profile of Mood States category scale (r=0.26). It should be noted that physical activity is not required to induce feelings of physical fatigue. Indeed, recent studies show that sitting and being sedentary for extended periods can contribute to feelings of fatigue (Ellingson et al., 2014). This effect may be exacerbated by cognitive work involving sustained attention.

After statistically controlling for variation in the prior night's sleep duration, dairy- and calorie-free brewed cocoa can acutely influence aspects of sustained attention. The caffeine in cocoa beverage with supplemented levels of caffeine enhances sustained attention, while the cocoa can attenuate the anxiety provoking side effects of the caffeine

The term “cocoa extract” used herein refers to a theobromine-containing sample sourced from a cacao bean. In general, these cocoa extracts have high relative theobromine content and preferably higher levels of theobromine than can be obtained from any aqueous extraction of a commercial cocoa powder. In addition, theobromine used in the compositions of the invention can be from any other source compatible with use as a food product. Also, combinations of cocoa extracts containing theobromine and theobromine from other sources can be used as a source of theobromine for the compositions of the invention. Similarly, caffeine used can be from any source compatible with use as a food product. Caffeine can be derived from green coffee extracts, cocoa extracts, or other sources.

The examples presented above and the contents of the application define and describe examples of the many combinations of caffeine and theobromine compositions, food products, and methods that can be produced or used according to the invention. None of the examples and no part of this description should be taken as a particular limitation on the scope of the invention as a whole.

What is claimed is:

1. A method of improving cognitive function in a subject as measured by improved alertness, sustained attention, or improvement in brain blood flow, comprising:
providing a food product having in a single dose approximately 70 mg of caffeine, 180 mg of theobromine, and 500 mg of cocoa-derived flavanols; and
administering the food product to a subject population of those with a daily coffee or caffeine intake of more than 400 mg caffeine daily.

2. The method of claim 1, wherein the food product is the only substantial source of caffeine and theobromine in the diet.

3. The method of claim 1, wherein the food product is a beverage.

4. A method of improving cognitive function in a subject as measured by improved alertness, sustained attention, or improvements in brain blood flow, comprising:
providing a food product having in a single dose approximately 23 mg of caffeine and 250 mg of cocoa-derived theobromine; and
administering the food product to a subject population of those with a daily coffee or caffeine habit of more than 400 mg caffeine per day.

5. The method of claim 4, wherein the food product is the only substantial source of caffeine and theobromine in the diet.

6. The method of claim 4, wherein the food product is a beverage.

7. The method of claim 4, wherein the theobromine is present as a cocoa extract.

8. An ingestible product for use in a treatment for reducing the anxiety side effects of coffee craving, the product consisting essentially of, in a single dose, approximately 23 mg of caffeine and 250 mg of theobromine.

9. The ingestible product of claim 8, wherein the theobromine is present as a cocoa extract.

10. The ingestible product of claim 8, wherein the product is a beverage.

11. The ingestible product of claim 8, further comprising one or more of the following: lowfat milk; sugar; cocoa; chocolate; maltodextrin; high fructose corn syrup; glycerin; green coffee extract; cocoa butter; corn syrup; cocoa processed with alkali; natural and artificial flavors; cellulose gel; milk fat; soy lecithin; lecithin; caramel color; lactose; carrageenan; vitamin B3 (Niacinamide); PGPR; an emulsifier; potassium sorbate; Vitamin 6 Pyridoxine (Pyridoxine Hydrochloride); xanthan gum; salt; mono- and di-glycerides; polysorbate 60; Vitamin B12 (Cyanocobalamin); Red 40 Color.

12. The method of claim 4, wherein the food product further comprises one or more of the following: lowfat milk; sugar; cocoa; chocolate; maltodextrin; high fructose corn syrup; glycerin; green coffee extract; cocoa butter; corn syrup; cocoa processed with alkali; natural and artificial flavors; cellulose gel; milk fat; soy lecithin; lecithin; caramel color; lactose; carrageenan; vitamin B3 (Niacinamide); PGPR; an emulsifier; potassium sorbate; Vitamin 6 Pyridoxine (Pyridoxine Hydrochloride); xanthan gum; salt; mono- and di-glycerides; polysorbate 60; Vitamin B12 (Cyanocobalamin); Red 40 color.

13. The method of claim 1, wherein the food product further comprises one or more of the following: lowfat milk; sugar; cocoa; chocolate; maltodextrin; high fructose corn syrup; glycerin; green coffee extract; cocoa butter; corn syrup; cocoa processed with alkali; natural and artificial flavors; cellulose gel; milk fat; soy lecithin; lecithin; caramel color; lactose; carrageenan; vitamin B3 (Niacinamide); PGPR; an emulsifier; potassium sorbate; Vitamin 6 Pyridoxine (Pyridoxine Hydrochloride); xanthan gum; salt; mono- and di-glycerides; polysorbate 60; Vitamin B12 (Cyanocobalamin); Red 40 color.
